(19) United States
(12) Patent Application Publication
(10) Pub. No.: US 2023/0360185 A1
(43) Pub. Date: Nov. 9, 2023

(54) IMAGE PROCESSING APPARATUS, IMAGE PROCESSING METHOD, AND NON-TRANSITORY COMPUTER-READABLE STORAGE MEDIUM

(71) Applicant: CANON KABUSHIKI KAISHA, Tokyo (JP)

(72) Inventors: Kosuke TERUI, Kanagawa (JP); Atsushi IWASHITA, Tokyo (JP); Ryuichi FUJIMOTO, Tokyo (JP)

(21) Appl. No.: 18/351,647

(22) Filed: Jul. 13, 2023

Related U.S. Application Data
(63) Continuation of application No. PCT/JP2021/048503, filed on Dec. 27, 2021.

(30) Foreign Application Priority Data
Mar. 3, 2021 (JP) 2021-033727

(51) Int. Cl. G06T 5/50; A61B 6/00

(52) U.S. Cl. CPC G06T 5/50; A61B 6/482; A61B 6/5258; G06T 2207/20224; G06T 2207/30004; G06T 2207/10116

(57) ABSTRACT

An image processing apparatus includes: a generation unit configured to generate a first image representing a thickness of a first material and a second image representing a thickness of a second material different from the first material using a plurality of images obtained based on a first combination of different radiation energies, and to generate a third image representing the thickness of the first material and a fourth image representing the thickness of the second material using a plurality of images obtained based on a second combination of different radiation energies; and an obtaining unit configured to obtain, using one of the first image and the second image and one of the third image and the fourth image, an enhanced image in which a third material different from the first material and the second material is enhanced.

### CROSS-REFERENCE TO RELATED APPLICATIONS

This application is a Continuation of International Patent Application No. PCT/JP2021/048503, filed Dec. 27, 2021, which claims the benefit of Japanese Patent Application No. 2021-033727, filed Mar. 3, 2021, both of which are hereby incorporated by reference herein in their entirety.

### BACKGROUND OF THE INVENTION

### Field of the Invention

The present invention relates to an image processing apparatus, an image processing method, and a non-transitory computer-readable storage medium and, more particularly, to an image processing apparatus suitably used for still image capturing such as general imaging or moving image capturing such as fluoroscopic imaging in medical diagnosis, an image processing method, and a non-transitory computer-readable storage medium.

### Background Art

A radiation imaging apparatus using a flat panel detector (to be abbreviated as an “FPD” hereinafter) made of a semiconductor material is currently widespread as an imaging apparatus used for medical image diagnosis or non-destructive inspection by X-rays. Such a radiation imaging apparatus is used as a digital imaging apparatus for still image capturing like general imaging or moving image capturing like fluoroscopic imaging in, for example, medical image diagnosis.

One of imaging methods using an FPD is energy subtraction. In energy subtraction, a plurality of images corresponding to X-rays of a plurality of different energies are obtained, and images of specific materials (for example, a bone image and a soft tissue image) are decomposed from the plurality of images using the difference between the X-ray attenuation rates of the materials. PTL 1 discloses a technique for smoothing the image of a soft tissue and subtracting the image from an accumulation image, thereby improving the quality of a bone image.

### CITATION LIST

### Patent Literature

PTL 1: Japanese Patent Laid-Open No. H03-285475

In an image of a soft tissue or a bone with a suppressed background, the contrast of a contrast agent or a medical device can change depending on the combination of radiation qualities of X-ray images before decomposition. Hence, to enhance the contrast agent or the medical device, X-ray images are preferably obtained by a combination of tube voltages with which the contrast is maximized.

However, in some cases, a radiation imaging apparatus cannot obtain an X-ray image by an optimum tube voltage because of a constraint such as an imaging environment. Even if imaging can be performed using an optimum tube voltage, it may be impossible to obtain a sufficient contrast.

The present invention provides to obtain an image with a predetermined material enhanced in a material decomposition image.

### SUMMARY OF THE INVENTION

According to one aspect of the present invention, there is provided an image processing apparatus comprising:
a generation unit configured to generate a first image representing a thickness of a first material and a second image representing a thickness of a second material different from the first material using a plurality of images obtained based on a first combination of different radiation energies, and to generate a third image representing the thickness of the first material and a fourth image representing the thickness of the second material using a plurality of images obtained based on a second combination of different radiation energies; and
an obtaining unit configured to obtain, using one of the first image and the second image and one of the third image and the fourth image, an enhanced image in which a third material different from the first material and the second material is enhanced.

Further features of the present invention will become apparent from the following description of exemplary embodiments with reference to the attached drawings.

### BRIEF DESCRIPTION OF THE DRAWINGS

FIG. 1 is a view showing an example of the configuration of a radiation imaging system according to the embodiment.

FIG. 2 is an equivalent circuit diagram of a pixel provided in a two-dimensional detector of an X-ray imaging apparatus.

FIG. 3 is a timing chart showing an operation of obtaining an X-ray image.

FIG. 4 is a timing chart for explaining energy subtraction processing.

FIG. 5 is a view showing the processing procedure of an image processing apparatus according to the first embodiment.

FIG. 6 is a view showing examples of material decomposition images.

FIG. 7 is a view showing the relationship between a contrast and a combination of X-ray energies.

FIG. 8 is a view showing examples of images in a case where an image operation is performed for images of the same material.

FIG. 9 is a view showing the processing procedure of an image processing apparatus according to the second embodiment.

FIG. 10 is a view showing examples of images in a case where an image operation is performed for images of different materials.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

Note that a radiation imaging apparatus (radiation imaging system) using X-rays as radiation will be described below. However, the present invention is not limited to this. Radiation according to the present invention includes not only α-rays, β-rays, and γ-rays that are beams generated by particles (including photons) emitted by radioactive decay but also beams having equal or more energy, for example, X-rays, particle rays, and cosmic rays.

### First Embodiment

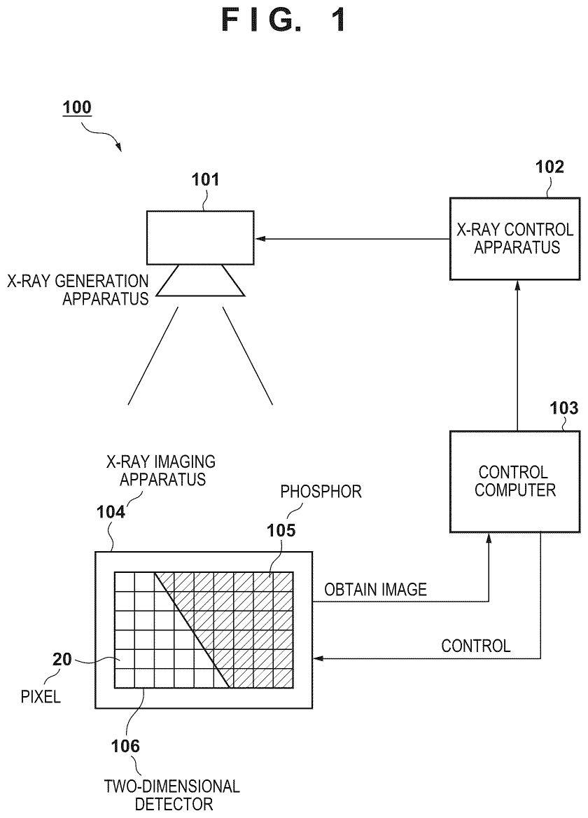

FIG. 1 is a block diagram showing an example of the configuration of a radiation imaging system 100 according to the first embodiment. The radiation imaging system 100 according to the first embodiment includes an X-ray generation apparatus 101, an X-ray control apparatus 102, a control computer 103, and an X-ray imaging apparatus 104.

The X-ray generation apparatus 101 performs X-ray irradiation. The X-ray control apparatus 102 controls X-ray irradiation by the X-ray generation apparatus 101. The control computer 103 controls the X-ray imaging apparatus 104 to obtain a radiation image (to be referred to as an X-ray image (image information) hereinafter) captured by the X-ray imaging apparatus 104. The control computer 103 functions as an image processing apparatus that performs image processing to be described later for the X-ray image obtained from the X-ray imaging apparatus 104. Note that the function of executing image processing may be imparted to the X-ray imaging apparatus 104. The X-ray imaging apparatus 104 is formed by a phosphor 105 that converts X-rays into visible light, and a two-dimensional detector 106 that detects the visible light. The two-dimensional detector 106 is a sensor in which pixels 20 configured to detect X-ray quanta are arranged in an array of X columns x Y rows, and outputs image information.

The control computer 103 includes a CPU as a hardware configuration, and executes programs stored in an internal storage unit (a ROM or a RAM), thereby controlling various kinds of operations of the control computer 103. For example, the CPU of the control computer 103 controls X-ray irradiation by the X-ray control apparatus 102 (X-ray generation apparatus 101) and X-ray image capturing operation by the X-ray imaging apparatus 104. In addition, the CPU implements various kinds of signal processing and image processing to be described later. Note that the operations of signal processing and image processing to be described later may be partially or wholly implemented by dedicated hardware. The internal storage unit stores programs to be executed by the CPU and various kinds of data, and stores radiation images (X-ray images) as a processing target. A display (not shown) can be connected to the control computer 103, and the display displays an image processed by image processing or performs various kinds of display under the control of the CPU.

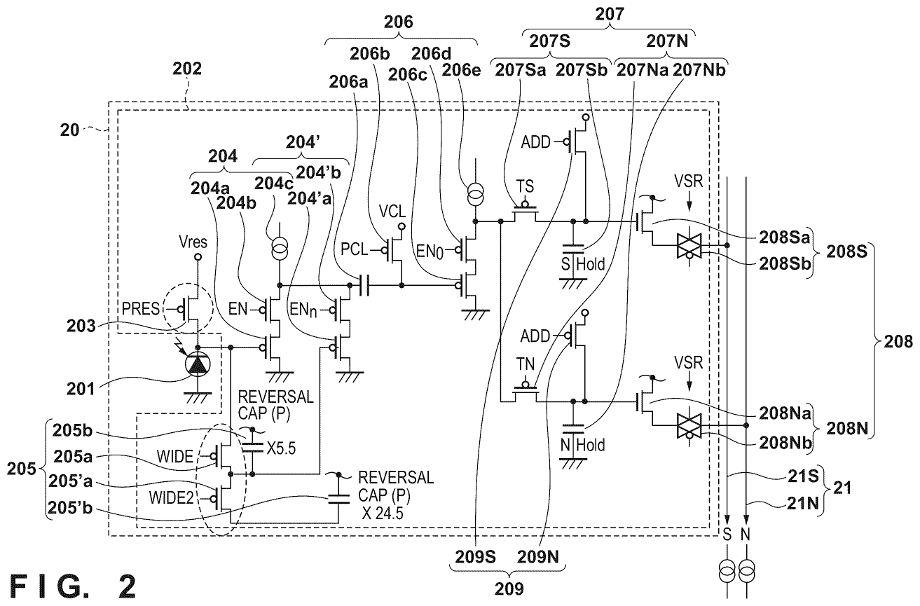

FIG. 2 is an equivalent circuit diagram of the pixel 20 included in the two-dimensional detector 106. The pixel 20 includes a photoelectric conversion element 201 and an output circuit unit 202. The photoelectric conversion element 201 can typically be a photodiode. The output circuit unit 202 includes an amplification circuit unit 204, a clamp circuit unit 206, a sample and hold circuit unit 207, and a selection circuit unit 208.

The photoelectric conversion element 201 includes a charge accumulation portion. The charge accumulation portion is connected to the gate of a MOS transistor 204a of the amplification circuit unit 204. The source of the MOS transistor 204a is connected to a current source 204c via a MOS transistor 204b. The MOS transistor 204a and the current source 204c form a source follower circuit. The MOS transistor 204b is an enable switch that is turned on when an enable signal EN supplied to its gate is set at an active level, and sets the source follower circuit in an operation state.

In the example shown in FIG. 2, the charge accumulation portion of the photoelectric conversion element 201 and the gate of the MOS transistor 204a form a common node, and this node functions as a charge-voltage converter that converts charges accumulated in the charge accumulation portion into a voltage. That is, a voltage V (=Q/C) determined by charges Q accumulated in the charge accumulation portion and a capacitance value C of the charge-voltage converter appears in the charge-voltage converter. The charge-voltage converter is connected to a reset potential Vres via a reset switch 203. When a reset signal PRES is set at an active level, the reset switch 203 is turned on, and the potential of the charge-voltage converter is reset to the reset potential Vres.

The clamp circuit unit 206 clamps, by a clamp capacitor 206a, noise output from the amplification circuit unit 204 in accordance with the reset potential of the charge-voltage converter. That is, the clamp circuit unit 206 is a circuit configured to cancel the noise from a signal output from the source follower circuit in accordance with charges generated by photoelectric conversion in the photoelectric conversion element 201. The noise includes kTC noise at the time of reset. Clamping is performed by turning on a MOS transistor 206b by setting a clamp signal PCL at an active level, and then turning off the MOS transistor 206b by setting the clamp signal PCL at an inactive level. The output side of the clamp capacitor 206a is connected to the gate of a MOS transistor 206c. The source of the MOS transistor 206c is connected to a current source 206e via a MOS transistor 206d. The MOS transistor 206c and the current source 206e form a source follower circuit. The MOS transistor 206d is an enable switch that is turned on when an enable signal ENO supplied to its gate is set at an active level, and sets the source follower circuit in an operation state.

The signal output from the clamp circuit unit 206 in accordance with charges generated by photoelectric conversion in the photoelectric conversion element 201 is written, as an optical signal, in a capacitor 207Sb via a switch 207Sa when an optical signal sampling signal TS is set at an active level. The signal output from the clamp circuit unit 206 when turning on the MOS transistor 206b immediately after resetting the potential of the charge-voltage converter is a clamp voltage. The noise signal is written in a capacitor 207Nb via a switch 207Na when a noise sampling signal TN is set at an active level. This noise signal includes an offset component of the clamp circuit unit 206. The switch 207Sa and the capacitor 207Sb form a signal sample and hold circuit 207S, and the switch 207Na and the capacitor 207Nb form a noise sample and hold circuit 207N. The sample and hold circuit unit 207 includes the signal sample and hold circuit 207S and the noise sample and hold circuit 207N.

When a driving circuit unit drives a row selection signal to an active level, the signal (optical signal) held in the capacitor 207Sb is output to a signal line 21S via a MOS transistor 208Sa and a row selection switch 208Sb. In addition, the signal (noise) held in the capacitor 207Nb is simultaneously output to a signal line 21N via a MOS transistor 208Na and a row selection switch 208Nb. The MOS transistor 208Sa forms a source follower circuit with a constant current source (not shown) provided on the signal line 21S. Similarly, the MOS transistor 208Na forms a source follower circuit with a constant current source (not shown) provided on the signal line 21N. The MOS transistor 208Sa and the row selection switch 208Sb form a signal selection circuit unit 208S, and the MOS transistor 208Na and the row selection switch 208Nb form a noise selection circuit unit 208N. The selection circuit unit 208 includes the signal selection circuit unit 208S and the noise selection circuit unit 208N.

The pixel 20 may include an addition switch 209S that adds the optical signals of the plurality of adjacent pixels 20. In an addition mode, an addition mode signal ADD is set at an active level, and the addition switch 209S is turned on. This causes the addition switch 209S to interconnect the capacitors 207Sb of the adjacent pixels 20, and the optical signals are averaged. Similarly, the pixel 20 may include an addition switch 209N that adds noise components of the plurality of adjacent pixels 20. When the addition switch 209N is turned on, the capacitors 207Nb of the adjacent pixels 20 are interconnected by the addition switch 209N, thereby averaging the noise components. An adder 209 includes the addition switches 209S and 209N.

The pixel 20 may include a sensitivity changing unit 205 for changing the sensitivity. The pixel 20 can include, for example, a first sensitivity change switch 205a, a second sensitivity change switch 205′a, and their circuit elements. When a first change signal WIDE is set at an active level, the first sensitivity change switch 205a is turned on to add the capacitance value of a first additional capacitor 205b to the capacitance value of the charge-voltage converter. This decreases the sensitivity of the pixel 20. When a second change signal WIDE2 is set at an active level, the second sensitivity change switch 205′a is turned on to add the capacitance value of a second additional capacitor 205′b to the capacitance value of the charge-voltage converter. This further decreases the sensitivity of the pixel 20. In this way, it is possible to receive a larger light amount by adding a function of decreasing the sensitivity of the pixel 20, thereby widening a dynamic range. When the first change signal WIDE is set at the active level, an enable signal ENw may be set at an active level to cause a MOS transistor 204′a to perform a source follower operation instead of the MOS transistor 204a.

The X-ray imaging apparatus 104 reads out the output of the pixel circuit as described above, causes an A/D converter (not shown) to convert the output into a digital value, and transfers the image to the control computer 103.

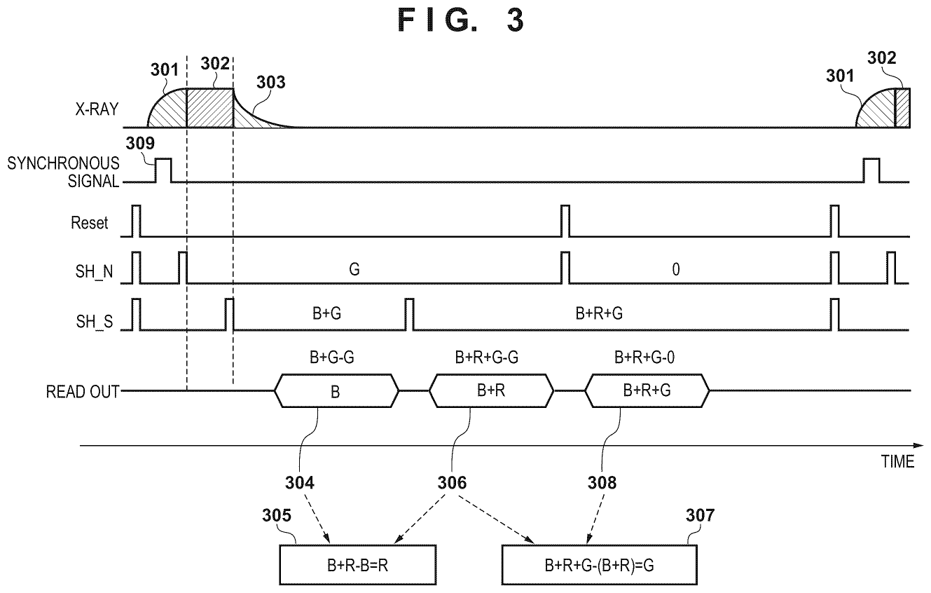

The operation of the radiation imaging system 100 (driving of the X-ray imaging apparatus 104) according to this embodiment will be described next. FIG. 3 is a view showing the driving timing when energy subtraction is performed in the radiation imaging system 100. When the abscissa represents the time, FIG. 3 shows timings of X-ray irradiation, a synchronous signal, reset of the photoelectric converting element 201, and readout of an image from the sample and hold circuit 207 and a signal line 21.

First, after the photoelectric converting element 201 is reset, X-ray irradiation is performed. The tube voltage of the X-rays ideally has a rectangular waveform but it takes a finite time for the tube voltage to rise or fall. Especially, if the time of irradiation of pulsed X-rays is short, the tube voltage is not considered to have a rectangular waveform any more, and has waveforms as shown in FIG. 3. That is, X-rays during the rising period, X-rays during the stable period, and X-rays during the falling period have different X-ray energies.

Hence, the noise sample and hold circuit 207N performs sampling after irradiation of X-rays 301 during the rising period, and the signal sample and hold circuit 207S performs sampling after irradiation of X-rays 302 during the stable period. After that, the difference between the signal lines 21N and 21S is read out as an image. At this time, a signal (G) of the X-rays 301 during the rising period is held in the noise sample and hold circuit 207N, and the sum (B+G) of the signal of the X-rays 301 during the rising period and a signal of the X-rays 302 during the stable period is held in the signal sample and hold circuit 207S. Therefore, an image 304 corresponding to the signal (B) of the X-rays 302 during the stable period is read out from the X-ray imaging apparatus 104.

Next, after completion of irradiation of X-rays 303 during the falling period and readout of the image 304, the signal sample and hold circuit 207S performs sampling again. After that, the difference between the signal lines 21N and 21S is read out as an image.

At this time, the signal (G) of the X-rays 301 during the rising period is held in the noise sample and hold circuit 207N, and the sum (B+R+G) of the signal of the X-rays 301 during the rising period, the signal of the X-rays 302 during the stable period, and the signal of the X-rays 303 during the falling period is held in the signal sample and hold circuit 207S

Hence, an image 306 corresponding to the signal (B) of the X-rays 302 during the stable period and the signal (R) of the X-rays 303 during the falling period is read out from the X-ray imaging apparatus 104.

After that, the photoelectric converting element 201 is reset, the noise sample and hold circuit 207N performs sampling again, and the difference between the signal lines 21N and 21S is read out as an image. At this time, a signal in a state in which irradiation of X-rays is not performed is held in the noise sample and hold circuit 207N, and the sum (B+R+G) of the signal of the X-rays 301 during the rising period, the signal of the X-rays 302 during the stable period, and the signal of the X-rays 303 during the falling period is held in the signal sample and hold circuit 207S. Therefore, an image 308 corresponding to the signal (G) of the X-rays 301 during the rising period, the signal (B) of the X-rays 302 during the stable period, and the signal (R) of the X-rays 303 during the falling period is read out.

After that, by calculating the difference between the images 306 and 304, an image 305 corresponding to the signal (R) of the X-rays 303 during the falling period is obtained. In addition, by calculating the difference between the images 308 and 306, an image 307 corresponding to the signal (G) of the X-rays 301 during the rising period is obtained.

The timing of resetting the sample and hold circuit 207 and the photoelectric converting element 201 is decided using a synchronous signal 309 indicating the start of irradiation of X-rays from the X-ray generation apparatus 101. As a method of detecting the start of irradiation of X-rays, a configuration for measuring the tube current of the X-ray generation apparatus 101 and determining whether the current value exceeds a preset threshold is suitably used.

Also, a configuration for repeatedly reading out the pixel 20 and determining whether the pixel value exceeds a preset threshold after completion of the reset of the photoelectric converting element 201 is suitably used. Alternatively, a configuration, incorporating an X-ray detector different from the two-dimensional detector 106 in the X-ray imaging apparatus 104, for determining whether a measured value of the X-ray detector exceeds a preset threshold is suitably used. In either method, after a time designated in advance elapses after the input of the synchronous signal 309, sampling of the signal sample and hold circuit 207S, sampling of the noise sample and hold circuit 207N, and reset of the photoelectric converting element 201 are performed.

As described above, the image 304 (corresponding to the signal (B)) corresponding to the stable period of the pulsed X-rays, the image 306 (corresponding to the signal (B+R)) corresponding to the sum of the signal during the rising period and that during the falling period, and the image 308 (corresponding to the signal (B+R+G)) corresponding to the sum of the signal during the rising period, that during the stable period, and that during the falling period are obtained. Since the energies of the X-rays irradiated when forming the three images are different, calculation is performed for the images, thereby making it possible to perform energy subtraction processing.

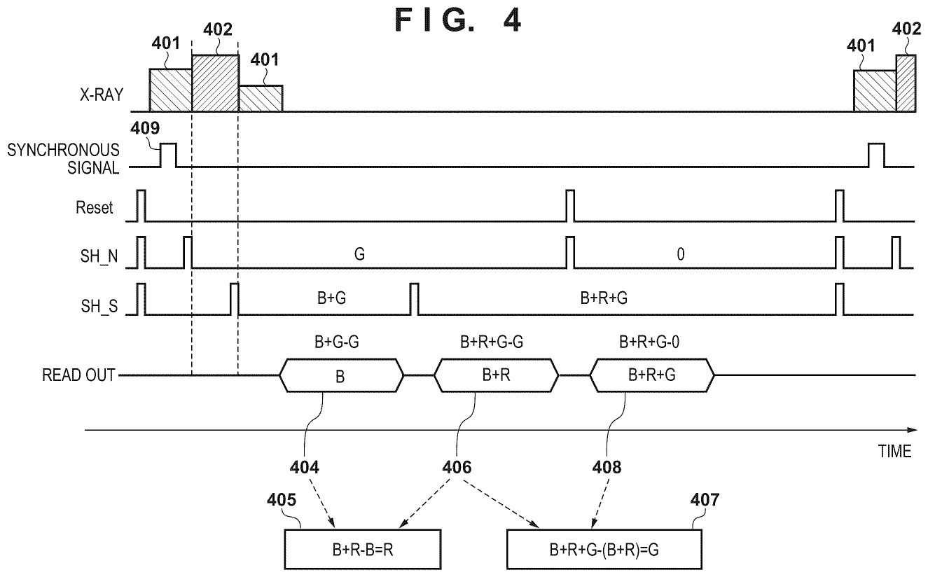

FIG. 4 shows the driving timing when energy subtraction is performed in the radiation imaging system 100 according to the first embodiment. The driving timing shown in FIG. 4 is different from the driving timing shown in FIG. 3 in that the tube voltage of the X-rays is actively switched.

First, after the reset of the photoelectric converting element 201, medium energy X-rays 401 are irradiated. After that, the noise sample and hold circuit 207N performs sampling, the tube voltage is switched to irradiate high energy X-rays 402, and the signal sample and hold circuit 207S then performs sampling. After that, the tube voltage is switched to irradiate low energy X-rays 403. Furthermore, the difference between the signal lines 21N and 21S is read out as an image. At this time, a signal (G) of the medium energy X-rays 401 is held in the noise sample and hold circuit 207N, and the sum (B+G) of the signal (G) of the medium energy X-rays 401 and a signal (B) of the high energy X-rays 402 is held in the signal sample and hold circuit 207S. Therefore, an image 404 corresponding to the signal (B) of the high energy X-rays 402 is read out from the X-ray imaging apparatus 104.

Next, after completion of the irradiation of the low energy X-rays 403 and the readout of the image 404, the signal sample and hold circuit 207S performs sampling again. After that, the difference between the signal lines 21N and 21S is read out as an image. At this time, the signal (G) of the medium energy X-rays 401 is held in the noise sample and hold circuit 207N, and the sum (B+R+G) of the signal (G) of the medium energy X-rays 401, the signal (B) of the high energy X-rays 402, and the signal (R) of the low energy X-rays 403 is held in the signal sample and hold circuit 207S. Therefore, an image 406 corresponding to the signal (B) of the high energy X-rays 402 and the signal (R) of the X-rays 403 during the falling period is read out from the X-ray imaging apparatus 104.

After that, the photoelectric converting element 201 is reset, the noise sample and hold circuit 207N performs sampling again, and the difference between the signal lines 21N and 21S is read out as an image. At this time, a signal in a state in which irradiation of X-rays is not performed is held in the noise sample and hold circuit 207N, and the sum (B+R+G) of the signal (G) of the medium energy X-rays 401, the signal (B) of the high energy X-rays 402, and the signal (R) of the low energy X-rays 403 is held in the signal sample and hold circuit 207S. Therefore, an image 408 corresponding to the signal (G) of the medium energy X-rays 401, the signal (B) of the high energy X-rays 402, and the signal (R) of the low energy X-rays 403 is read out from the X-ray imaging apparatus 104.

After that, by calculating the difference between the images 406 and 404, an image 405 corresponding to the signal (R) of the low energy X-rays 403 is obtained. In addition, by calculating the difference between the images 408 and 406, an image 407 corresponding to the signal (G) of the medium energy X-rays 401 is obtained. A synchronous signal 409 is similar to in FIG. 3. When an image is thus obtained while actively switching the tube voltage, the energy difference between X-ray images can be made larger as compared to the method shown in FIG. 3. Note that the order of X-ray energies can be changed. For example, the X-rays 401 may have low energy, the X-rays 402 may have high energy, and the X-rays 403 may have medium energy.

The control computer 103 obtains a radiation image (X-ray image (image information)) captured by the X-ray imaging apparatus 104. The control computer 103 performs various kinds of processing for the X-ray image obtained from the X-ray imaging apparatus 104. Energy subtraction processing according to this embodiment is divided into three stages of correction processing, signal processing, and image processing. The processing of each stage will be described below.

First, an image is obtained without X-ray irradiation to the X-ray imaging apparatus 104 by the driving shown in FIG. 3 or 4. This image is an image corresponding to the fixed pattern noise (FPN) of the X-ray imaging apparatus 104. The fixed pattern noise (FPN) component is removed by subtracting the component of the image. This correction is called offset correction.

Next, imaging is performed by irradiating X-rays to the X-ray imaging apparatus 104 in a state in which there is no object, thereby obtaining an image (X-ray image) by the driving shown in FIG. 3 or 4. An image (white image) obtained by offset-correcting the X-ray image is prepared, and the X-ray image is divided by the white image, thereby evenly correcting the variation of the characteristic such as the sensitivity to the pixel 20. This correction is called gain correction. At this time, if the correction target image and the white image are obtained under the same X-ray irradiation conditions, the image after the gain correction is an image of an attenuation rate I/I₀.

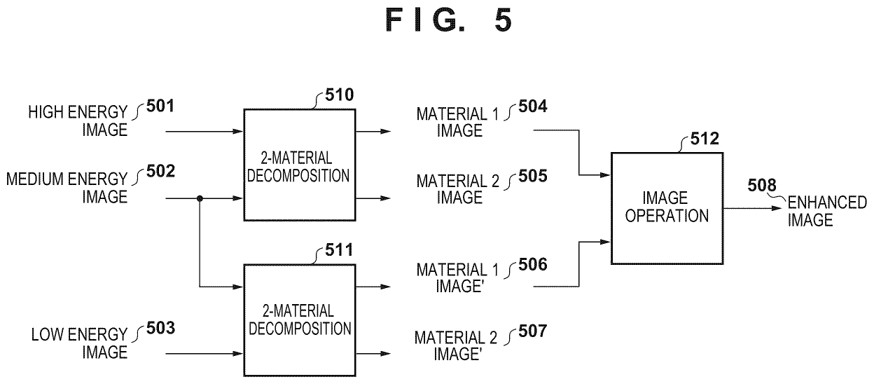

FIG. 5 is a view showing the processing procedure of an image processing apparatus according to the first embodiment. The control computer 103 generates a first image (to be referred to as a material 1 image 504 hereinafter) representing the thickness of a first material and a second image (to be referred to as a material 2 image 505 hereinafter) representing the thickness of a second material, which are decomposed from a plurality of images (501 and 502) obtained based on a first combination of different radiation energies. Also, the control computer 103 generates a third image (to be referred to as a material 1 image′ 506 hereinafter) representing the thickness of the first material and a fourth image (to be referred to as a material 2 image′ 507 hereinafter) representing the thickness of the second material, which are decomposed from a plurality of images (502 and 503) obtained based on a second combination of different radiation energies.

The plurality of images obtained based on the first combination of different radiation energies include an image (to be referred to as the high energy image 501 hereinafter) captured at a first energy and an image (to be referred to as the medium energy image 502 hereinafter) captured at a second energy lower than the first energy. The plurality of images obtained based on the second combination of different radiation energies include an image (medium energy image 502) captured at the second energy and an image (to be referred to as the low energy image 503 hereinafter) captured at a third energy lower than the second energy.

The high energy image 501, the medium energy image 502, and the low energy image 503 are images after offset correction and gain correction are performed for X-ray images obtained by the driving shown in FIG. 3 or 4. In processing blocks 510 and 511 of 2-material decomposition, the thickness images of the first material (to be referred to as “material 1” hereinafter) and the second material (to be referred to as “material 2” hereinafter) are obtained from two images of different energies. For the sake of convenience, of the two images, the image of the higher energy is defined as an image H, and the image of the lower energy is defined as an image L. Defining material 1 and material 2 as a soft tissue and a bone, respectively, a case where a soft tissue thickness image S and a bone thickness image B are obtained will be described. Let μ_{S}(E) be the linear attenuation coefficient of the soft tissue at an energy E, μ_{B}(E) be the linear attenuation coefficient of the bone at the energy E, N_{H}(E) be a high energy X-ray spectrum, and N_{L}(E) be a low energy X-ray spectrum. By solving nonlinear simultaneous equations (1) below, the bone thickness B and the soft tissue thickness S can be obtained.

$$\begin{matrix}H = \frac{{\int }_{0}^{∞} {N}_{H} ( E ) exp \{ - {μ}_{S} ( E ) S - {μ}_{B} ( E ) B \} EdE}{{\int }_{0}^{∞} {N}_{H} ( E ) EdE} \\ L = \frac{{\int }_{0}^{∞} {N}_{L} ( E ) exp \{ - {μ}_{S} ( E ) S - {μ}_{B} ( E ) B \} EdE}{{\int }_{0}^{∞} {N}_{L} ( E ) EdE}\end{matrix} \qquad ( 1 )$$

The X-ray spectra N_{H}(E) and N_{L}(E) are obtained by simulation or actual measurement. The linear attenuation coefficient μ_{B}(E) of the bone at the energy E and the linear attenuation coefficient μ_{S}(E) of the soft tissue at the energy E are obtained from a database of NIST (National Institute of Standards and Technology) or the like. Note that to solve equations (1), the Newton-Raphson method is may be used, or an iterative method such as a least square method or a bisection method may be used. Also, a configuration for generating a table by obtaining, in advance, the soft tissue thicknesses S and the bone thicknesses B for various combinations of the attenuation rates H at high energy and the attenuation rates L at low energy, and obtaining the soft tissue thickness S and the bone thickness B at high speed by referring to this table may be used.

The material 1 image 504 and the material 2 image 505 are material decomposition images obtained by performing 2-material decomposition for the high energy image 501 and the medium energy image 502. In addition, the material 1 image′ 506 and the material 2 image′ 507 are material decomposition images obtained by performing 2-material decomposition for the medium energy image 502 and the low energy image 503.

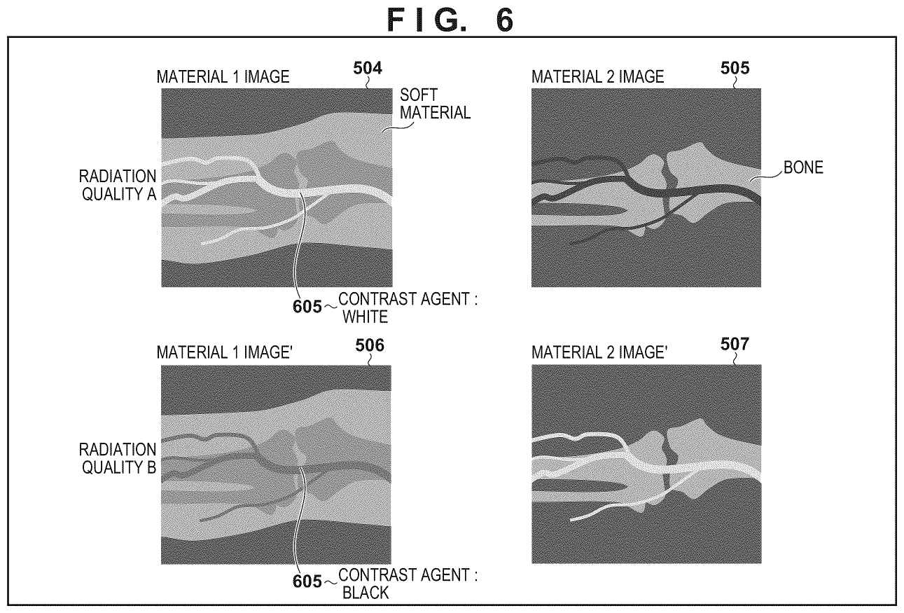

FIG. 6 is a view showing examples of material decomposition images, and shows examples of the material 1 image 504, the material 2 image 505, the material 1 image′ 506, and the material 2 image′ 507. The object is a lower limb (knee part), and a contrast agent is injected into blood vessels. In the images, three materials, that is, a soft material (soft tissue) such as a muscle or fat, a bone, and a contrast agent exist. The material 1 image 504 and the material 1 image′ 506 are soft tissue thickness images, and the material 2 image 505 and the material 2 image′ 507 are bone thickness images. The muscle and fat appear only in the soft tissue thickness images, and the bone appears only in the bone thickness images. On the other hand, the contrast agent appears in both the soft tissue thickness images and the bone thickness images. The contrast agent never appears in only one of these because the attenuation coefficient of the contrast agent that is a third material (to be referred to as “material 3” hereinafter) is not included in equations (1) and is converted into the soft tissue thickness and the bone thickness at a predetermined ratio (depending on the X-ray energy). Note that the contrast of the bone appears in the soft tissue thickness image because there is a decrease of the bone thickness. If 2-material decomposition is accurately performed, the values of a region without the contrast agent match between the soft tissue thickness images (the material 1 image 504 and the material 1 image′ 506) and the bone thickness images (the material 2 image 505 and the material 2 image′ 507). On the other hand, the values of the region of the contrast agent do not match. This is because when the X-ray energy is changed, the ratio of converting the thickness of the contrast agent into the thickness of the soft tissue and the thickness of the bone changes. Note that the thickness of the region of the contrast agent may take a negative value depending on the energy.

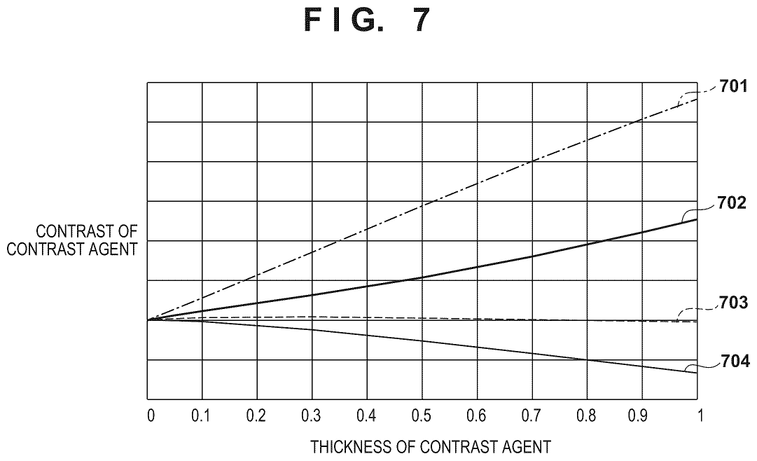

FIG. 7 is a view showing the relationship between a contrast and a combination of X-ray energies, and shows a graph concerning the contrast of the contrast agent in the thickness images of material 1, which are obtained by changing the combination of X-ray energies. The abscissa of the graph represents the thickness of the contrast agent, and the ordinate represents the contrast of the contrast agent. If the combination of X-ray energies changes, like combinations 701 and 702, the contrast changes (changes in the positive direction). There may be no contrast, like a combination 703 (there is no change). The positive/negative state of the contrast may change, like a combination 704 (changes in the negative direction).

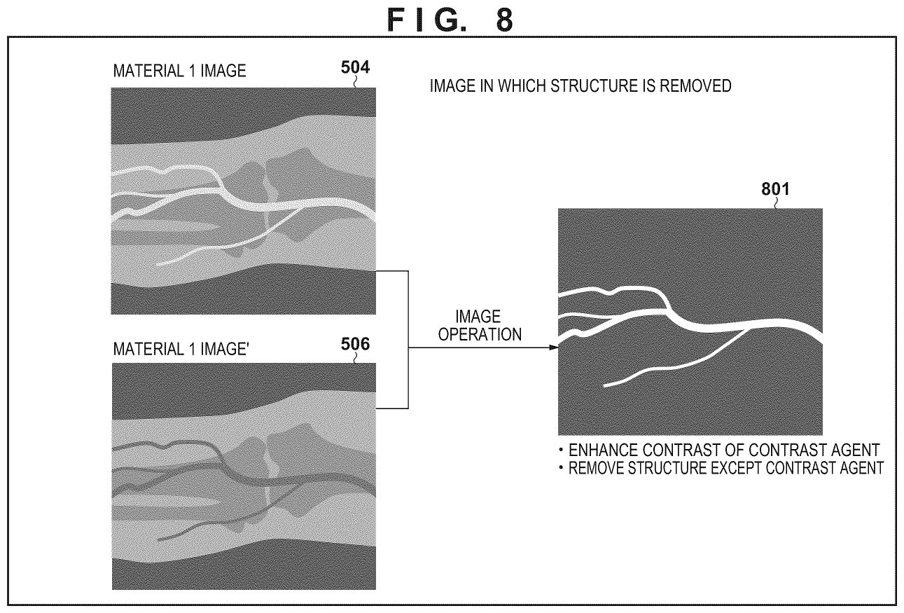

FIG. 8 is a view showing examples of images in a case where an image operation is performed for images of the same material, and shows examples of images obtained by, in the processing procedure shown in FIG. 5, obtaining the material 1 image 504 and the material 1 image′ 506 as material decomposition images based on the combination 701 (first combination) of X-ray energies and the combination 704 (second combination) of X-ray energies, and performing an image operation 512. Here, the material 1 image 504 and the material 1 image′ 506 are soft tissue thickness images. The control computer 103 performs the image operation 512 of subtracting image information based on the material 1 image 504 and the material 1 image′ 506, thereby obtaining an enhanced image 801 (enhanced image 508 (FIG. 5)) in which the contrast of material 3 (contrast agent) is enhanced.

In the image 801, the contrast of the contrast agent is enhanced (becomes high) as compared to the thickness images before processing by the image operation 512 because the positive/negative state of the contrast of the contrast agent is different between the material 1 image 504 and the material 1 image′ 506 (white in the material 1 image 504 shown in FIG. 6, and black in the material 1 image′ 506). In addition, since the thickness of material 1 (soft tissue) is canceled between the material 1 image 504 and the material 1 image′ 506, only the contrast agent can be seen (processing close to maskless DSA can be done by performing the operation in moving images). Hence, since the contrast of the contrast agent improves, and the structures of the soft tissue and the bone are removed, visibility may improve. By the image operation 512, it is possible to obtain the image 801 in which the contrast of material 3 (contrast agent) is enhanced, and the soft tissue and the bone are removed.

Note that in this embodiment, the processing of performing an image operation for images of the same material is not limited to this example. The image operation 512 can also be performed, concerning the bone thickness image, based on the material 2 image 505 and the material 2 image′ 507. In this case as well, it is possible to obtain the image 801 in which the contrast of material 3 (contrast agent) is enhanced, and the soft tissue and the bone are removed.

### Second Embodiment

Processing of a radiation imaging system 100 according to the second embodiment will be described next. An example of the configuration of the radiation imaging system 100 is similar to the configuration described in the first embodiment, and a repetitive description will be omitted.

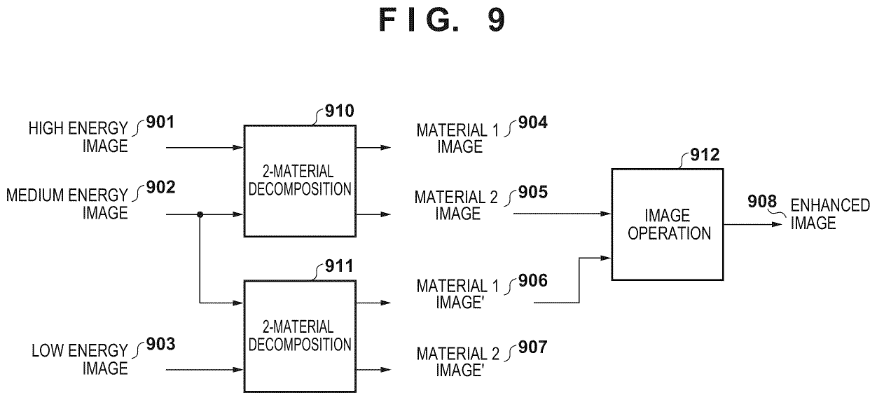

FIG. 9 is a view showing the processing procedure of an image processing apparatus according to the second embodiment. A control computer 103 generates a first image (to be referred to as a material 1 image 904 hereinafter) representing the thickness of a first material and a second image (to be referred to as a material 2 image 905 hereinafter) representing the thickness of a second material, which are decomposed from a plurality of images (901 and 902) obtained based on a first combination of different radiation energies. Also, the control computer 103 generates a third image (to be referred to as a material 1 image′ 906 hereinafter) representing the thickness of the first material and a fourth image (to be referred to as a material 2 image′ 907 hereinafter) representing the thickness of the second material, which are decomposed from a plurality of images (902 and 903) obtained based on a second combination of different radiation energies.

Here, the plurality of images obtained based on the first combination of different radiation energies include an image (to be referred to as the high energy image 901 hereinafter) captured at a first energy and an image (to be referred to as the medium energy image 902 hereinafter) captured at a second energy lower than the first energy. The plurality of images obtained based on the second combination of different radiation energies include an image (medium energy image 902) captured at the second energy and an image (to be referred to as the low energy image 903 hereinafter) captured at a third energy lower than the second energy.

The high energy image 901, the medium energy image 902, and the low energy image 903 are images after offset correction and gain correction are performed for X-ray images obtained by the driving shown in FIG. 3 or 4. In processing blocks 910 and 911 of 2-material decomposition, the thickness images (904 and 906) of material 1 and the thickness images (905 and 907) of material 2 are obtained from two images of different energies.

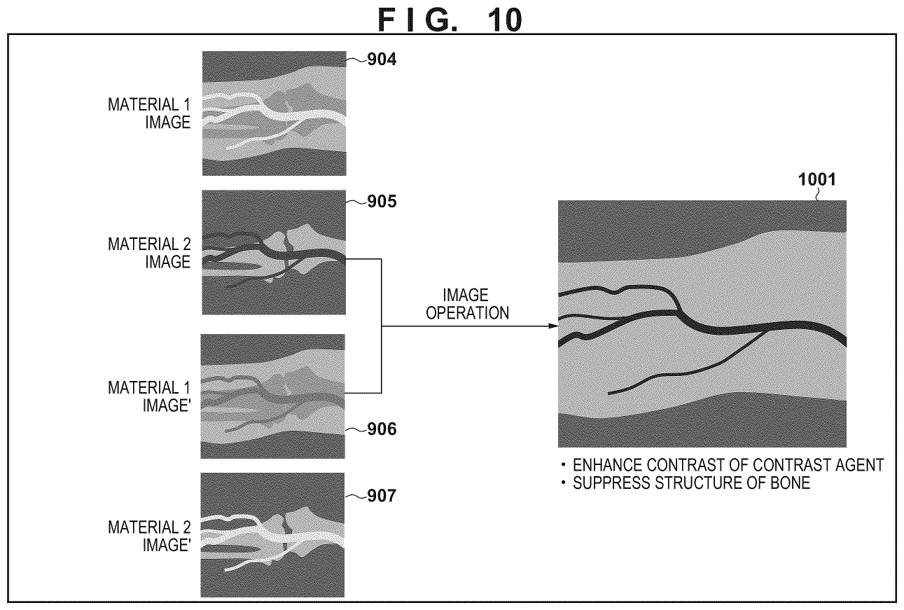

FIG. 10 is a view showing examples of images in a case where an image operation is performed for images of different materials. In the examples of images shown in FIG. 10 (the material 1 image 904, the material 2 image 905, the material 1 image′ 906, and the material 2 image′ 907), the object is a lower limb (knee part), and a contrast agent is injected into blood vessels. In the images, three materials, that is, a soft material (soft tissue) such as a muscle or fat, a bone, and a contrast agent exist. The material 1 image 904 and the material 1 image′ 906 are soft tissue thickness images, and the material 2 image 905 and the material 2 image′ 907 are bone thickness images. The muscle and fat appear only in the soft tissue thickness images, and the bone appears only in the bone thickness images. On the other hand, the contrast agent appears in both the soft tissue thickness images and the bone thickness images. The contrast agent never appears in only one of these because the attenuation coefficient of the contrast agent that is material 3 is not included in equations (1) and is converted into the soft tissue thickness and the bone thickness at a predetermined ratio (depending on the X-ray energy). Note that the contrast of the bone appears in the soft tissue thickness image because there is a decrease of the bone thickness. If 2-material decomposition is accurately performed, the values of a region without the contrast agent match between the soft tissue thickness images (the material 1 image 904 and the material 1 image′ 906) and the bone thickness images (the material 2 image 905 and the material 2 image′ 907). On the other hand, the values of the region of the contrast agent do not match. This is because when the X-ray energy is changed, the ratio of converting the thickness of the contrast agent into the thickness of the soft tissue and the thickness of the bone changes. Note that the thickness of the region of the contrast agent may take a negative value depending on the energy.

When the thickness image of material 1 and the thickness image of material 2 are added, it is possible to remove the structure of the bone in the soft material and improve the visibility of the contrast agent (bone backfilling image). However, as described above, the thickness of the contrast agent is converted into the soft tissue thickness and the bone thickness at a predetermined ratio. That is, if the thickness of the material image is large, the thickness of the other image becomes small. Hence, even if the thickness image of material 1 and the thickness image of material 2 are added, a sufficient contrast to the background material may not be obtained (addition of contrast white/black). In this embodiment, images with a large contrast agent thickness or images with a small contrast agent thickness are added. That is, the control computer 103 adds the image information of the material 1 image 904 and the material 2 image′ 907 (adds contrast white/white) or adds the image information of the material 2 image 905 and the material 1 image′ 906 (adds contrast black/black), thereby obtaining an image 1001 (enhanced image 908 (FIG. 9)) in which the contrast of material 3 (contrast agent) is enhanced.

The image 1001 shown in FIG. 10 is an example of an image when the material 2 image 905 and the material 1 image′ 906 are added. The contrast of the contrast agent is enhanced (becomes high) as compared to the thickness images before processing by the image operation 912 because the positive/negative states of the contrast of the contrast agent match between the material 2 image 905 and the material 1 image′ 906 (the images both have a small contrast agent thickness, and the contrast is black). In addition, since the thickness of material 2 (bone) is backfilled by adding the material 2 image 905 (bone) and the material 1 image′ 906 (soft tissue), only the soft tissue and the contrast agent, which have a continuous thickness, can be seen. Hence, since the contrast of the contrast agent improves, and the structure of the bone is removed, visibility may improve. By the image operation 912, it is possible to obtain the image 1001 in which the contrast of material 3 (contrast agent) is enhanced, and the bone is removed.

Note that if the X-ray irradiation, the driving, and the processing described with reference to FIGS. 3 to 10 are continuously repeated, a moving image can be created. Also, if the processing is executed at a high speed, real-time display can be performed. As the plurality of images (501 and 502, or 901 and 902) obtained based on the first combination of radiation energies, the control computer 103 obtains images obtained by performing a sample and hold operation a plurality of times during one shot of radiation irradiation and generates a first image (the material 1 image 504 or 904) and a second image (the material 2 image 505 or 905).

Also, as the plurality of images (502 and 503, or 902 and 903) obtained based on the second combination of radiation energies, the control computer 103 obtains images obtained by performing a sample and hold operation a plurality of times during one shot of radiation irradiation and generates a third image (the material 1 image′ 506 or 906) and a fourth image (the material 2 image′ 507 or 907). Then, the control computer 103 can perform display control for displaying, on the display unit, an enhanced image obtained by the operation of image information based on the generated images as a moving image or in real time.

In the first and second embodiments, the first material includes at least water, fat, or a soft material that does not contain calcium, and the second material includes at least calcium, hydroxyapatite, or bone. In the first and second embodiments, a case where the third material (material 3) is a contrast agent has been described. In addition to this example, the embodiments can also be applied to a material containing a metal, like a medical device (a stent, a catheter, a guide wire, or the like).

When performing an operation of a thickness image, noise may be reduced by filter processing using a spatial filter. Before an operation of image information is performed, the control computer 103 can perform noise reduction processing by applying a spatial filter to a thickness image to be used for the operation.

Also, an unnecessary component by a soft tissue or a bone can removed by multiplying a coefficient (correction coefficient) before an image operation (subtraction or addition) based on a thickness image is performed. Before the operation of image information is performed, the control computer 103 can perform image processing for removing the component of a predetermined tissue included in a thickness image by multiplying the thickness image to be used for the operation by a correction coefficient.

It is also possible to do adjustment to make a structure by a soft tissue or a bone visible. For example, before an operation of image information is performed, the control computer 103 can perform display control and image processing for enhancing the component of a predetermined tissue included in a thickness image to be used for the image operation and displaying the component on the display unit.

As the combination (for example, the combination 701 or 704 shown in FIG. 7) of X-ray energies, a combination that makes the contrast of a contrast agent large in an image after subtraction of a thickness image is preferably selected. The larger the difference of the attenuation rate of the contrast agent to X-ray energy is, the more easily the contrast is imparted. Hence, it is preferable that the average energy of at least one image in X-ray images is lower than the k-edge of iodine.

Threshold determination may be performed for the image 801 or 1001 after contrast enhancement to determine the presence/absence of a contrast agent, and enhancement by image processing may be performed for the region of the contrast agent. The control computer 103 determines a region where a contrast agent (material 3) exists depending on whether the pixel value of the image 801 or 1001 (enhanced image) after contrast enhancement exceeds a preset threshold, and performs image processing of enhancing the region where the contrast agent exists and displaying it on the display unit. As the enhancement of the region, for example, the region corresponding to the contrast agent may be enhanced by coloring and displayed. Alternatively, enhanced display may be performed by fixing the pixel value of the region corresponding to the contrast agent to a specific value. Enhanced display may be performed by generating a difference from other regions by multiplying the pixel value of the region corresponding to the contrast agent by a coefficient. The enhancement by image processing is not limited to the image 801 or 1001 after contrast enhancement and can also be applied to any of an X-ray image, a thickness image, and a thickness image after an operation. The control computer 103 can determine a region where the thickness changes between a plurality of thickness images to be used for an operation of image information (image operation 512 or 912) as a region where a contrast agent (third material) exists, and perform image processing of enhancing the region in the plurality of thickness images and the image 801 or 1001 (enhanced image) after contrast enhancement and displaying it on the display unit.

In the first and second embodiments, a method of decomposing two sets of two materials from X-ray images of three energies and performing an operation has been described. However, a 2-material decomposition image may be created for the third set, and the operation (subtraction or addition of image information) may be performed for the three images. Images obtained by performing addition or subtraction for X-ray images may be used for 2-material decomposition.

In the first and second embodiments, the X-ray imaging apparatus 104 is an indirect type X-ray sensor using a phosphor. However, the embodiment of the present invention is not limited to this form. For example, a direct type X-ray sensor using a direct conversion material such as CdTe may be used.

Also, in the first and second embodiments, the tube voltage of the X-ray generation apparatus 101 is changed. However, the embodiment of the present invention is not limited to this form. For example, the energy of X-rays irradiated to the X-ray imaging apparatus 104 may be changed by temporally switching the filter of the X-ray generation apparatus 101.

Also, in the first and second embodiments of the present invention, the X-ray energy is changed, thereby obtaining an image of a different energy. However, the embodiment of the present invention is not limited to this form. For example, a stacked configuration may be used, in which a plurality of phosphors 105 and two two-dimensional detectors 106 (sensors) are overlaid, thereby obtaining images of different energies from the two-dimensional detector on the front side and the two-dimensional detector on the rear side with respect to the direction of incidence of X-rays.

Also, in the first and second embodiments, energy subtraction processing is performed using the control computer 103 of the radiation imaging system 100. However, the embodiment of the present invention is not limited to this form. An image obtained by the control computer 103 may be transferred to another computer, and energy subtraction processing may be performed. For example, an obtained image may be transferred to another computer (image viewer) via a medical PACS, and the processing result may be displayed after energy subtraction processing is performed.

In the above-described embodiments, the control computer 103 directly obtains an image from the X-ray imaging apparatus 104 and performs energy subtraction processing. However, the present invention is not limited to this. An image (a still image or a moving image) captured by the X-ray imaging apparatus 104 may be stored in an external storage device, and the control computer 103 may read out the image from the storage device and perform energy subtraction processing.

As described above, according to the above-described embodiments, it is possible to provide an image processing technique (image processing apparatus) or a radiation imaging system capable of obtaining an image with a predetermined material enhanced in a material decomposition image.

According to the present invention, it is possible to obtain an image with a predetermined material enhanced in a material decomposition image. It is therefore possible to provide an image in which the visibility of a contrast agent or a medical device is improved.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a generation unit configured to generate a first image representing a thickness of a first material and a second image representing a thickness of a second material different from the first material using a plurality of images obtained based on a first combination of different radiation energies, and to generate a third image representing the thickness of the first material and a fourth image representing the thickness of the second material using a plurality of images obtained based on a second combination of different radiation energies; and
an obtaining unit configured to obtain, using one of the first image and the second image and one of the third image and the fourth image, an enhanced image in which a third material different from the first material and the second material is enhanced.

2. The image processing apparatus according to claim 1, wherein
the plurality of images obtained based on the first combination include an image captured at a first energy and an image captured at a second energy lower than the first energy, and
the plurality of images obtained based on the second combination include the image captured at the second energy and an image captured at a third energy lower than the second energy.

3. The image processing apparatus according to claim 2, wherein
the generation unit generates the first image and the second image by processing of material decomposition based on the image captured at the first energy and the image captured at the second energy, and
generates the third image and the fourth image by processing of material decomposition based on the image captured at the second energy and the image captured at the third energy.

4. The image processing apparatus according to claim 1, wherein the obtaining unit obtains the enhanced image by performing subtraction of image information based on a plurality of images representing a thickness of the same material.

5. The image processing apparatus according to claim 1, wherein the obtaining unit obtains the enhanced image by performing subtraction of image information based on the first image representing the thickness of the first material and the third image representing the thickness of the first material.

6. The image processing apparatus according to claim 1, wherein the obtaining unit obtains the enhanced image by performing subtraction of image information based on the second image representing the thickness of the second material and the fourth image representing the thickness of the second material.

7. The image processing apparatus according to claim 1, wherein the obtaining unit obtains the enhanced image by performing addition of image information based on a plurality of images representing thicknesses of different materials.

8. The image processing apparatus according to claim 1, wherein the obtaining unit obtains the enhanced image by performing addition of image information based on the first image representing the thickness of the first material and the fourth image representing the thickness of the second material.

9. The image processing apparatus according to claim 1, wherein the obtaining unit obtains the enhanced image by performing addition of image information based on the second image representing the thickness of the second material and the third image representing the thickness of the first material.

10. The image processing apparatus according to claim 1, wherein before an operation of image information is performed, the obtaining unit multiplies a thickness image to be used for the operation by a correction coefficient, thereby removing a component of a predetermined tissue included in the thickness image.

11. The image processing apparatus according to claim 10, wherein before the operation of the image information is performed, the obtaining unit performs image processing of enhancing the component of the predetermined tissue included in the thickness image to be used for the operation and displaying the component on a display unit.

12. The image processing apparatus according to claim 10, wherein before the operation of the image information is performed, the obtaining unit performs noise reduction processing by applying a spatial filter to the thickness image to be used for the operation.

13. The image processing apparatus according to claim 1, wherein the obtaining unit determines a region where the third material exists depending on whether a pixel value of the enhanced image exceeds a preset threshold, and performs image processing of enhancing the region and displaying the region on a display unit.

14. The image processing apparatus according to claim 10 wherein
the obtaining unit determines a region where the thickness changes between a plurality of thickness images to be used for the operation of the image information as a region where a third material exists, and
performs image processing of enhancing the region in the plurality of thickness images and the enhanced image and displaying the region on a display unit.

15. The image processing apparatus according to claim 1, wherein
the generation unit obtains, as the plurality of images obtained based on the first combination, images obtained by performing a sample and hold operation a plurality of times during one shot of radiation irradiation and generates the first image and the second image,
obtains, as the plurality of images obtained based on the second combination, images obtained by performing a sample and hold operation a plurality of times during one shot of radiation irradiation and generates the third image and the fourth image, and
the obtaining unit displays, on a display unit, the enhanced image obtained by the operation of image information input from the generation unit as a moving image or in real time.

16. The image processing apparatus according to claim 1, wherein the first material includes at least water or fat, the second material includes at least calcium, hydroxyapatite, or bone, and the third material includes a contrast agent or a material containing a metal.

17. An image processing apparatus comprising:
an obtaining unit configured to obtain, using (a) a material decomposition image obtained by processing of material decomposition using information relating to a first combination of different radiation energies and (b) a material decomposition image obtained by the processing of the material decomposition using information relating to a second combination of different radiation energies, an enhanced image in which a material different from a target of the material decomposition is enhanced.

18. The image processing apparatus according to claim 1, wherein
an average energy of a radiation spectrum at which at least one image of the plurality of images obtained based on the first combination of the radiation energies is obtained is an energy lower than a k-edge of iodine, and
an average energy of a radiation spectrum at which at least one image of the plurality of images obtained based on the second combination of the radiation energies is obtained is an energy lower than the k-edge of iodine.

19. An image processing method comprising
obtaining, using (a) a material decomposition image obtained by processing of material decomposition using information relating to a first combination of different radiation energies and (b) a material decomposition image obtained by the processing of the material decomposition using information relating to a second combination of different radiation energies, an enhanced image in which a material different from a target of the material decomposition is enhanced.

20. A non-transitory computer-readable storage medium storing a program for causing a computer to execute the radiation imaging method according to claim 19.
